(19) United States
(12) Patent Application Publication
(10) Pub. No.: US 2025/0304609 A1
(43) Pub. Date: Oct. 2, 2025

(54) POLYMORPHIC FORM OF CHALCONE GLYCOSIDE COMPOUND, AND PREPARATION METHOD AND APPLICATION THEREOF

(71) Applicant: ZHEJIANG YONGNING PHARMACEUTICAL CO., LTD., Taizhou (CN)

(72) Inventors: Ben CAI, Taizhou (CN); Tianjian YE, Taizhou (CN); Wei ZHANG, Taizhou (CN); Guanzan JIANG, Taizhou (CN); Jindan WANG, Taizhou (CN); Min LU, Taizhou (CN); Meijuan GUO, Taizhou (CN); Shenjie PAN, Taizhou (CN); Yongling CHEN, Taizhou (CN); Wei HUANG, Taizhou (CN); Zhuoqi XIE, Taizhou (CN)

(21) Appl. No.: 18/864,485

(22) PCT Filed: Dec. 25, 2023
(86) PCT No.: PCT/CN2023/141373

§ 371 Date: Nov. 8, 2024

(30) Foreign Application Priority Data
Dec. 30, 2022 (CN) 202211743871.3

(51) Int. Cl. C07H 7/04; A61K 31/7034; A61P 7/02; A61P 9/10

(52) U.S. Cl. CPC C07H 7/04; A61K 31/7034; A61P 7/02; A61P 9/10

(57) ABSTRACT

The present invention discloses a polymorphic form of a chalcone glycoside compound, and a preparation method and an application thereof in a medicament for treating a vascular disease. The chalcone glycoside compound has a structure of the general formula below or a hydrate thereof, where R₁ and R₂ are β-D-glucosyl or α-D-glucosyl; R₃ is hydrogen or hydroxyl; and M is a metal ion, and n is 1-2. The chalcone glycoside compound described in the present invention can effectively inhibit platelet aggregation, has a protective effect against cell apoptosis, can significantly relieve ischemia/ischemia-reperfusion injury, increases a serum SOD content, and improves behavior disorders and tissue infarction focuses induced by ischemia-reperfusion. Moreover, this crystal form has a simple process, high purity and excellent stability, and has important significance for developing medicaments for treating vascular diseases with stronger therapeutic effects, lower toxicity and urgent clinical needs.

### FIELD OF TECHNOLOGY

The present invention belongs to the field of pharmacy, and specifically relates to a polymorphic form, an amorphous form and a mixed crystal of a chalcone glycoside compound, and a preparation method and an application thereof.

### BACKGROUND

Chalcone or its derivatives can scavenge a large amount of free radicals generated during ischemia-reperfusion injury and inflammatory response, inhibit lipid peroxidation, and thus protect the myocardium or nerves from damage. On the other hand, chalcone is an antagonist of platelet-activating factors (PAF), which can inhibit platelet aggregation and inflammatory response induced by PAF, and at the same time inhibit platelet aggregation induced by adenosine diphosphate sodium salt (ADP), significantly prolong coagulation time and prothrombin time, reduce a fibrinogen content, and thus inhibit thrombosis.

Current drug research and development is mainly based on the understanding of the pathogenesis and pathophysiology of diseases. At present, the complexity of vascular diseases urgently requires the development of new therapeutic drugs. The time from treatment to vascular recanalization should be shortened as much as possible, so as to minimize the infarct area and fully protect neurological function on the basis of restoring blood circulation in ischemic brain tissue. The combined application of neuroprotection and thrombolysis is crucial for the treatment of vascular diseases. On the one hand, reperfusion may be beneficial in promoting the use of neuroprotective agents in ischemic tissue. On the other hand, neuroprotection should counteract the harmful effects of reperfusion injury by inhibiting inflammation and oxidative stress of vascular nerve units, thus improving the efficacy and safety of thrombolysis. The research and development and clinical translation of neuroprotective agents have always been the focus of attention in the field of treatment.

Many compounds have polymorphic forms. Different crystal forms have their own physical structures, stability, and solubility, which in turn affect their chemical stability and pharmacokinetic properties. Therefore, the study of crystal forms plays a very important role in the development of drugs.

### SUMMARY

The present invention provides a polymorphic form of a chalcone glycoside compound and an application thereof in preparation of a medicament for treating a vascular disease. The present invention studies the pharmacological effects of chalcone glycoside compounds in antioxidation and antiplatelet aggregation, and conducts pharmacological tests for the treatment of ischemia-reperfusion loss.

A chalcone glycoside compound, having a structure of:

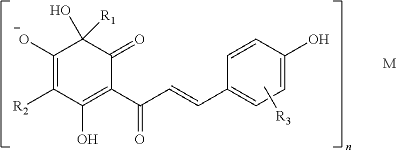

or a hydrate thereof:

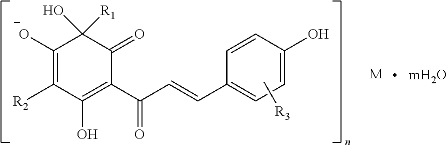

where R₁ and R₂ are β-D-glucosyl or α-D-glucosyl; R₃ is hydrogen or hydroxyl; and M is a metal ion, and n is 1-2.

A method for preparing the above-mentioned chalcone glycoside compound, where the chalcone glycoside compound is obtained by reacting an intermediate free acid of the chalcone glycoside compound with an alkaline compound of M.

The alkaline compound of M is a carbonate or a hydroxide.

Preferably, n is 2.

Preferably, M is Na⁺ or Ca²⁺.

Preferably, m is 0.5-20.

Preferably, the chalcone glycoside compound has a structure of:

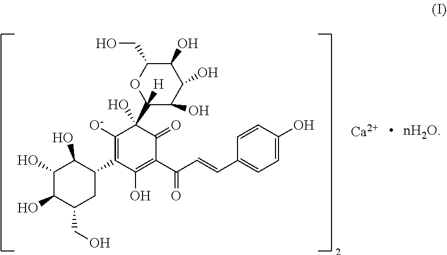

Alternatively, the chalcone glycoside compound has a structure of:

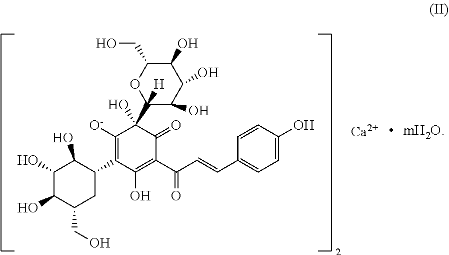

The compound shown in the formula (II) can be obtained by salification of an intermediate free acid thereof with calcium carbonate or calcium hydroxide.

Preferably, m=0.5-20; and further preferably, m=1-17.

Furthermore, the compound further has an octahedral ligand stereostructure.

Furthermore, the compound is: (S)-3,6-dihydroxy-4-((E)-3-(4-hydroxyphenyl) acryloyl)-5-oxo-6-((2R,3R,4S,5S,6R)-3,4,5-trihydroxy-6-(hydroxymethyl)tetrahydro-2H-pyran-2-yl)-2-((2S,3R,4R,5S,6R)-3,4,5-trihydroxy-6-(hydroxymethyl)tetrahydro-2H-pyran-2-yl)cyclohexyl-1,3-diene-1-hydroxycalcium.

Furthermore, the compound is: a(S)-3,6-dihydroxy-4-((E)-3-(4-hydroxyphenyl) acryloyl)-5-oxo-6-((2R,3R,4S,5S,6R)-3,4,5-trihydroxy-6-(hydroxymethyl)tetrahydro-2H-pyran-2-yl)-2-((2S,3R,4R,5S,6R)-3,4,5-trihydroxy-6-(hydroxymethyl)tetrahydro-2H-pyran-2-yl)cyclohexyl-1,3-diene-1-hydroxycalcium-hydrate.

A chalcone glycoside compound, having a crystal form A, a crystal form B, an amorphous form, or a mixed crystal form.

A crystal form A of a chalcone glycoside compound, having the structure shown in the formula (I) or (II), where an XRPD spectrum has diffraction peaks at 2θ=7.234 degrees, 7.866 degrees, 8.243 degrees, 8.792 degrees, 14.251 degrees, 15.791 degrees, and 16.283 degrees, and the 2θ deviation is ±0.2 degrees.

Preferably, the XRPD spectrum further has diffraction peaks at 2θ=9.959 degrees, 10.716 degrees, 12.722 degrees, 16.786 degrees, 20.764 degrees, and 23.955 degrees.

Further preferably, the XRPD spectrum of the crystal form A is shown in FIG. 1.

Preferably, the crystal form A is a yellow crystalline powder.

A crystal form B of a chalcone glycoside compound, having the structure described in the above formula (I) or (II), and having an orthorhombic crystal system, P212121 (19 #) space group, and unit cell parameters of a=14.3241(8) Å, b=21.9523(11) Å, c=22.3923(15) Å, V=7041.2(7) Å³, and Z=4.

Further preferably, the crystal form B is a yellow needle-shaped crystal.

An amorphous form of a chalcone glycoside compound, having the structure described in the above formula (I) or (II), and having an amorphous form. The XRPD spectrum has no characteristic X-ray powder diffraction peaks.

Preferably, the amorphous compound is an orange powder.

A mixed crystal of a chalcone glycoside compound, having a mixed crystal form of two or more of the above-mentioned crystal form A, crystal form B and amorphous form.

Preferably, the crystal form A is a dominant crystal form.

A method for preparing a crystal form A of a chalcone glycoside compound, including two types of:
a first type: in a water or methanol-water system, salifying and crystallizing an intermediate free acid of the chalcone glycoside compound to directly obtain the crystal form A; and
a second type: adopting water or methanol-water as a recrystallization solvent, dissolving the chalcone glycoside compound, and then crystallizing to obtain the A crystal form.

Preferably, during recrystallization, the chalcone glycoside compound is dissolved in hot water (with a concentration of 50 mg/ml-250 mg/ml) or dissolved in methanol, then water is added until a volume ratio of methanol to water reaches 4:1-1:3, and crystallization is performed to obtain the crystal form A.

A method for preparing a crystal form B of a chalcone glycoside compound, including: adopting methanol/tetrahydrofuran as a recrystallization solvent, dissolving the chalcone glycoside compound, and then adopting a solvent diffusion method to obtain the crystal form B.

Preferably, a volume ratio of methanol/tetrahydrofuran is 1:1-1:9.

A method for preparing an amorphous form of a chalcone glycoside compound, including: adopting ethanol as a recrystallization solvent, dissolving the chalcone glycoside compound, and then crystallizing to obtain the amorphous form.

A method for preparing a mixed crystal of a chalcone glycoside, including: adopting mechanical pulverization to obtain the mixed crystal, or adopting ethanol-water as a recrystallization solvent, dissolving the chalcone glycoside compound, and then crystallizing to obtain the mixed crystal.

An application of a chalcone glycoside compound in preparation of a medicament for treating a vascular disease, where the chalcone glycoside compound is the chalcone glycoside compound according to any one of the above-mentioned technical solutions.

Preferably, the disease that the anti-vascular disease medicament is used to treat is a vascular disease caused by free radical oxidation, inflammatory mediators and platelet aggregation.

As a preferred option, the vascular disease includes one or more of stroke, a coronary syndrome, a pulmonary heart disease, a peripheral vascular disease, and peripheral neuropathy.

The polymorphic compound described in the present invention can be prepared by extracting effective parts from traditional Chinese medicine, followed by crystallization through cation exchange resin, further salification and crystallization, and solvent recrystallization, etc.

In vitro test: the platelet aggregation effect induced by ADP in rats shows that the chalcone glycoside compound of the present invention can effectively inhibit platelet aggregation, and especially in the medium and high dose groups, the chalcone glycoside compound can significantly inhibit the maximum platelet aggregation rate induced by ADP (p<0.001). For the effect of protecting H9c2 cardiomyocytes from oxygen-glucose deprivation/reperfusion injury, the chalcone glycoside compound of the present invention has a significant inhibitory effect on cell apoptosis. In vivo test: 24 h after the ischemia-reperfusion model group rats are modeled, the chalcone glycoside compound of the present invention can significantly increase the content of SOD in serum, and improve behavior disorders and brain tissue infarction focuses induced by cerebral ischemia-reperfusion in rats.

The beneficial effects of the present invention are that the novel chalcone crystal form of the present invention is a natural active compound with a novel structure reported for the first time, and has significant pharmacological effects of free radical scavenging and antiplatelet aggregation and a pharmacodynamic effect of reducing reperfusion injury. Moreover, this crystal form exhibits excellent stability, and has important significance for developing medicaments for treating vascular diseases with stronger therapeutic effects, lower toxicity and urgent clinical needs.

### BRIEF DESCRIPTION OF THE DRAWINGS

FIG. 1 is a typical XRD spectrum of a crystal type A;

FIG. 2 is a polarizing microscope photograph of a single crystal;

FIG. 3 is a typical XRD spectrum of an amorphous form;

FIG. 4 is a typical XRD spectrum of a mixed crystal;

FIG. 5 shows an effect of YN17 compound on rat platelet aggregation in Example 2;

FIG. 6 shows a protective effect of YN17 on cellular oxygen-glucose deprivation/reperfusion injury;

FIG. 7 shows an effect of YN17 on behavior disorders in rats with cerebral ischemia-reperfusion; and

FIG. 8 shows an effect of YN17 on cerebral tissue infarction in rats with cerebral ischemia-reperfusion.

### DESCRIPTION OF THE EMBODIMENTS

The following examples are used to further illustrate the present invention. It should be understood that examples of the present invention are merely illustrative of the present invention and are not intended to limit the present invention.

### Example 1: Preparation Method and Structural Characterization of a Polymorphic Form of a Chalcone Glycoside Compound

Preparation of a polymorphic form of a chalcone glycoside compound: 500 g of a starting material (safflower yellow, purity 88.64%, Yongning Pharma active pharmaceutical ingredient approval number (Z20050145) was prepared into a solution of 200 mg/mL, and passed through a hydrogen type cation exchange resin, with a sample loading flow rate of 20 cm/min. After the sample loading was completed, purified water was used for elution, the penetration liquid was collected and placed for crystallization, suction filtration was performed under reduced pressure, and the filter cake was drip washed with an appropriate amount of purified water, dried under reduced pressure, pulverized, and passed through a 50-mesh sieve to prepare an intermediate (a free acid of the chalcone glycoside compound), with a yield of 396 g (79.2%) and purity of 96.82%. The intermediate was added to a glass-lined reaction kettle, prepared into a suspension of 175 mg/ml by addition of water for injection, and added with calcium carbonate (with a molar amount of 0.55 of the free acid of the chalcone glycoside compound) (or calcium hydroxide can also be adopted) while being stirred. When the yellow suspension turned into an orange-red solution and the pH rose to about 6.0, stirring was stopped, the solution was filtered through a 0.22 μm filter membrane and then placed for crystallization, suction filtration was performed under reduced pressure, and the filter cake was drip washed with an appropriate amount of water for injection and dried under reduced pressure to obtain a YN17 sample ((S)-3,6-dihydroxy-4-((E)-3-(4-hydroxyphenyl) acryloyl)-5-oxo-6-((2R,3R,4S,5S,6R)-3,4,5-trihydroxy-6-(hydroxymethyl)tetrahydro-2H-pyran-2-yl)-2-((2S,3R,4R,5S,6R)-3,4,5-trihydroxy-6-(hydroxymethyl)tetrahydro-2H-pyran-2-yl)cyclohexyl-1,3-diene-1-hydroxycalcium, denoted as a calcium salt), with a total yield of 337 g yellow solid (67.4%), crystal form A, and purity of 99.75%.

Preparation of a crystal form B sample: the above crystal form A sample was taken and added to a flask, and added with methanol/tetrahydrofuran (with a volume ratio of 1:1-1:9), and a solvent diffusion method was carried out to obtain a yellow needle-shaped crystal (the solvent slowly evaporated);

Preparation of an amorphous form: the above crystal form A sample was taken and added to a flask, added with ethanol to prepare a solution with a concentration of 20 mg/ml-60 mg/ml, heated in an oil bath, stirred and dissolved under reflux, filtered, allowed to stand for cooling and crystallization, and subjected to suction filtration, and the filter cake was dried under vacuum to obtain an orange powder; and

Preparation of a mixed crystal: the above crystal form A sample was taken and mechanically pulverized, or the sample was added with ethanol water (with a volume ratio of 2:1-1:2), dissolved under reflux, filtered, allowed to stand for cooling and crystallization, and subjected to suction filtration, and the filter cake was dried under vacuum to obtain an orange to yellow solid.

Preparation of other salt forms: the intermediate (the free acid of the chalcone glycoside compound) was taken, added with deionized water to prepare a suspension of about 200 mg/ml, added with 0.5 mol of carbonate under stirring for reaction for 5-30 minutes, filtered and subjected to gel column chromatography, and eluted with water. The eluent was collected, concentrated, filtered with a 0.22 μm filter membrane, and then freeze-dried to obtain a loose lump, which is amorphous (where the sodium salt was prepared with sodium carbonate, labeled YN17-ST).

For the calcium salt, in addition to the above preparation method in Example 1, each crystal form can also be prepared by adopting the following recrystallization method:
Crystal form A: the sample (crystal form A or crystal form B or amorphous form or mixed crystal) was dissolved in hot water (with a concentration of 50 mg/ml-250 mg/ml) or dissolved with methanol, then added with water until the volume ratio of methanol-water reached 4:1-1:3, filtered, allowed to stand for cooling and crystallization, and subjected to suction filtration, and the filter cake was dried under vacuum to obtain a yellow solid;
Preparation of a crystal form B sample: the sample (crystal form A or amorphous form or mixed crystal) was added to a flask, and dissolved by addition of methanol/tetrahydrofuran, and a solvent diffusion method was carried out to obtain a yellow needle-shaped crystal (the solvent slowly evaporated);
Preparation of an amorphous form: the sample (crystal form A or crystal form B or mixed crystal) was added to a flask, added with ethanol, heated in an oil bath, stirred and dissolved under reflux, filtered, allowed to stand for cooling and crystallization, and subjected to suction filtration, and the filter cake was dried under vacuum to obtain an orange powder; and
Preparation of a mixed crystal: the crystal form A or crystal form B or amorphous form or mixed crystal samples were mechanically pulverized, or the sample was added with ethanol-water, dissolved under reflux, filtered, allowed to stand for cooling and crystallization, and subjected to suction filtration, and the filter cake was dried under vacuum to obtain an orange to yellow solid.

The crystal form A of the calcium salt was the dominant crystal form (1) From the stability comparison data of salt forms in Table 1 and Table 2, it can be seen that the crystal form A was very stable, while the crystal forms of other salt forms were amorphous, and the relevant substances and contents were easily degraded. (2) From the perspective of process development, the crystal form A had a crystalline system of water, a low equipment requirement, high purity, and a high yield. However, other crystal forms or amorphous forms required organic solvents or lyophilizers, which were not ideal. (3) When the chalcone glycoside compound was a calcium salt, there were multiple stable crystal forms, while other salt forms were amorphous, making it difficult to obtain stable crystal forms, which further illustrated the advantages of calcium salts.

The structural characterization data of the chalcone glycoside compound prepared were as follows:
(S)-3,6-dihydroxy-4-((E)-3-(4-hydroxyphenyl) acryloyl)-5-oxo-6-((2R,3R,4S,5S,6R)-3,4,5-trihydroxy-6-(hydroxymethyl)tetrahydro-2H-pyran-2-yl)-2-((2S,3R,4R,5S,6R)-3,4,5-trihydroxy-6-(hydroxymethyl)tetrahydro-2H-pyran-2-yl)cyclohexyl-1,3-diene-1-hydroxycalcium.

FTIR(KBr) vₘₐₓ cm⁻¹: 3384, 2938, 2893, 1647, 1636, 1605, 1516, 1447, 1236, 1081, 929, 834;

¹H NMR (500 MHz, DMSO-d6): δ 9.78 (s, 1H), 7.40 (m, 3H), 7.31 (d, J=16.0 Hz, 1H), 6.77 (d=18.5 Hz, 2H), 4.95 (m, 1H), 4.84 (m, 2H), 4.73 (d=5.5 Hz, 1H), 4.70 (d=4.5 Hz, 1H), 4.63 (m, 1H), 4.45 (m, 1H), 421 (d=9.5 Hz, 1H), 4.13 (m, 1H), 4.04 (d=5.0 Hz, 1H), 3.68-3.61 (m, 3H), 3.35 (m, 3H) 3.12-2.96 (m, 6H);

¹³C NMR (500 MHZ, DMSO-d6): δ202.9, 194.4, 189.7, 184.5, 180.6, 159.1, 137.3, 130.1, 127.5, 123.0, 116.2, 105.9, 100.2, 86.1(86.0), 81.3, 80.0, 79.8, 78.7, 74.0, 71.3, 69.6, 69.4, 62.2, 60.4;

HRMS (ESI): m/z calcd for [C₂₇H₃₁O₁₆]⁻: 611.12.

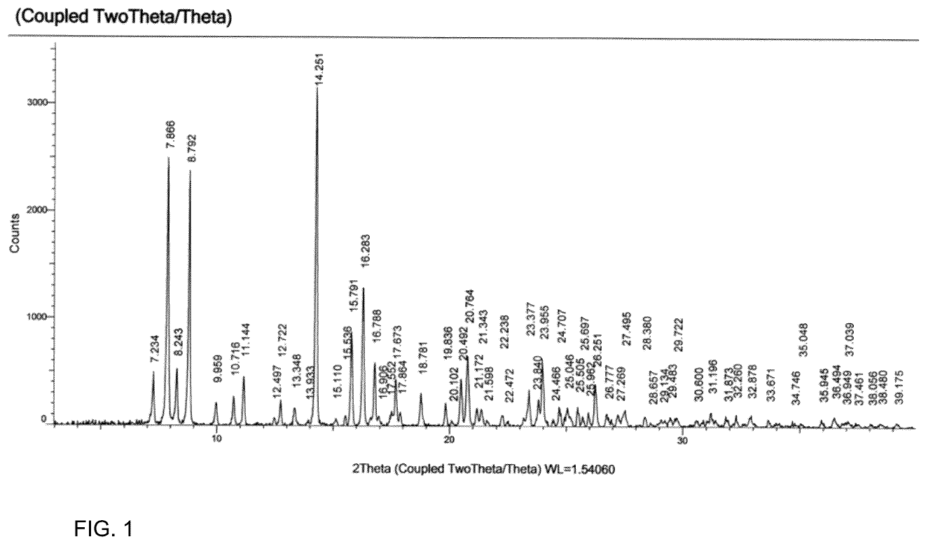

Crystal form A: the XRPD spectrum had diffraction peaks at 7.234, 7.866, 8.243, 8.792, 14.251, 15.791, 16.283, and their deviation ±0.2 degrees. (FIG. 1)

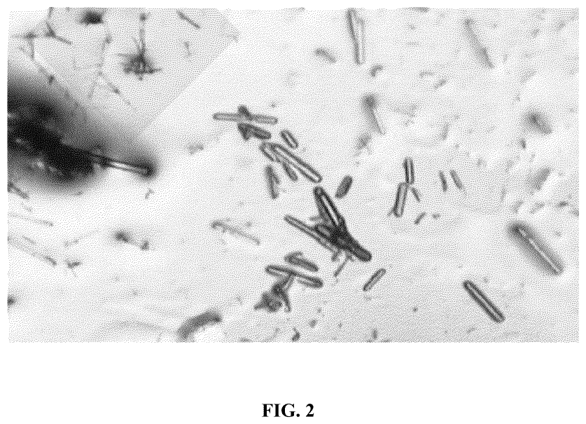

Crystal form B: orthorhombic crystal system, space group P2₁2₁2₁ (19 #), and unit cell parameters: a=14.3241(8) Å, b=21.9523(11) Å, c=22.3923(15) Å, V=7041.2(7) Å3, and Z=4. (FIG. 2)

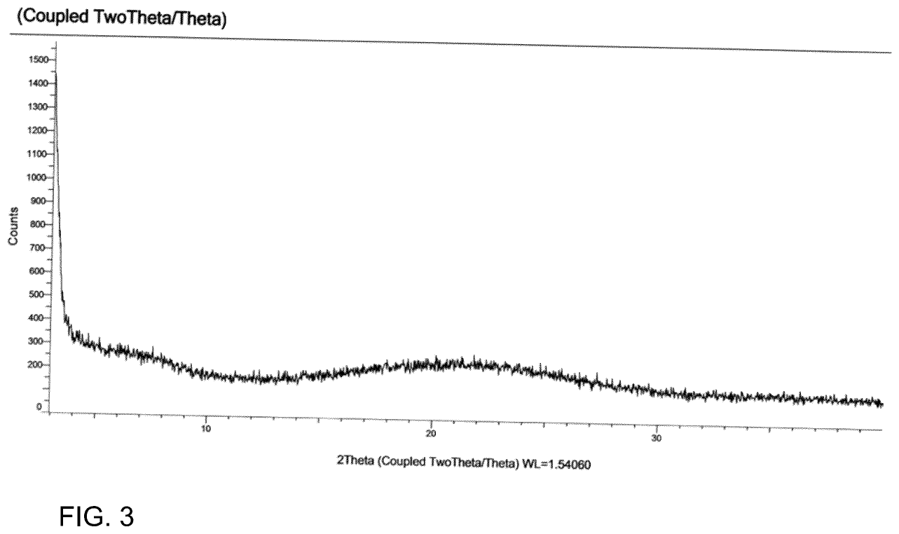

Crystal form C: the XRPD spectrum had no diffraction peaks. (FIG. 3)

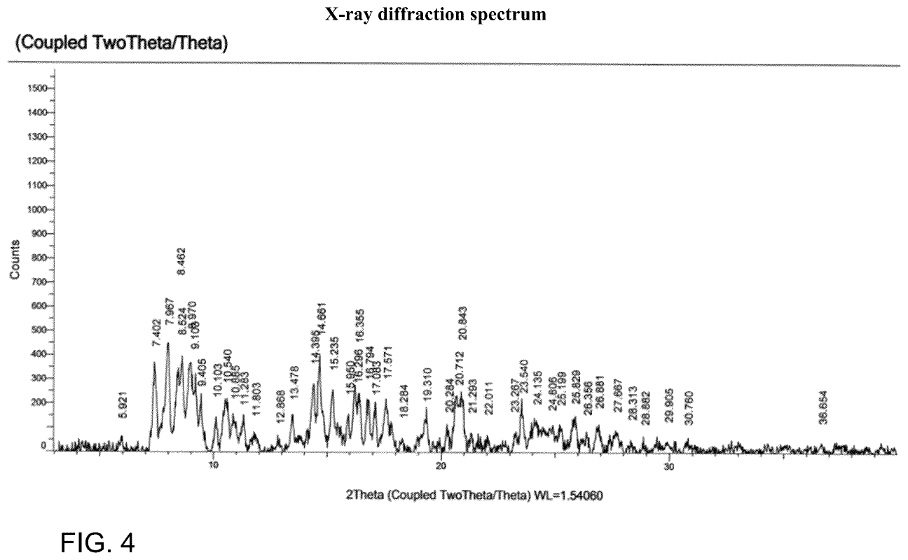

Mixed crystal: the XRPD spectrum had diffraction peaks at 7.261, 7.829, 8.259, 8.770, 14.187, 15.769, 16.258, and their deviation ±0.2 degrees. (FIG. 4)

The crystal form A for the stability data in the following table is prepared by the method of Example 1. The intermediate was adopted, water was used as a system, calcium carbonate was added for reaction, the solution was filtered and then allowed to stand for crystallization, and subjected to suction filtration, and the filter cake was washed with water and dried by suction, and dried under reduced pressure at 30° C. for 4-6 hours to obtain the crystal form A.

The stability data of the crystal form A are shown in the following table:

TABLE 1
Stability data of the crystal form A

| | | Accelerated | Long-term |
|---|---|---|---|
| Crystal form A | 0 months | 6 months | 6 months |
| Character | Yellow | Yellow | Yellow |
| | crystalline | crystalline | crystalline |
| | powder | powder | powder |
| pH | 5.9 | 5.8 | 5.9 |
| Relevant | Single impurity | Single impurity | Single impurity |
| substance | 0.25% | 0.25% | 0.25% |
| | Total impurities | Total impurities | Total impurities |
| | 0.25% | 0.54% | 0.32% |
| Water content | 9.9% | 10.1% | 10.7% |
| Calcium | 3.19% | 3.26% | 3.21% |
| element | | | |
| Content | 97.0% | 96.5% | 96.7% |
| determination | | | |

For stability tests and inspection methods, please see the attached stability solutions and quality standards.

TABLE 2
Stability data of other salt types (represented
by the sodium salt with the best stability data)

| Amorphous | | Accelerated | Long-term |
|---|---|---|---|
| form | 0 months | 6 months | 6 months |
| Character | Yellow | Yellow | Yellow |
| | loose lump | loose lump | loose lump |
| pH | 6.0 | 6.8 | 6.4 |
| Relevant | Single impurity | Single impurity | Single impurity |
| substance | 0.36% | 6.41% | 1.86% |
| | Total impurities | Total impurities | Total impurities |
| | 0.95% | 9.53% | 3.02% |
| Water content | 2.3% | 7.3% | 5.7% |
| Content | 95.3% | 81.5% | 91.7% |
| determination | | | |

### Example 2: Evaluation of the Antiplatelet Aggregation Effect of a Chalcone Glycoside Compound

The present invention evaluated the ability of test compounds to prolong clotting time, inhibit thrombosis and improve blood supply by measuring the inhibitory ability of the chalcone glycoside compound to ADP-induced platelet aggregation. The present invention used clopidogrel as a positive control, and the experimental results were based on the platelet aggregation inhibition rate as a parameter.

Preparation of platelets. The centrifuge tubes with the collected blood samples were placed in a centrifuge, the samples were centrifuged at 160 g for 10 minutes at room temperature, and the supernatant was taken to obtain initial platelet rich plasma (iPRP), which was stored at room temperature for later use. The remaining portion was centrifuged at 2000 g for 10 minutes, and then the supernatant was taken to obtain platelet poor plasma (PPP).

The platelet count was measured by using a fully automatic blood cell analyzer to detect iPRP and PPP. An appropriate volume of PPP and iPRP was mixed to obtain PRP platelets, and the platelet count was 2.5-3×10¹¹/L.

Determination of a platelet aggregation rate. 300 μl of PPP was taken in a sample cup for zero setting; then 270 μl of PRP platelets was taken, 30 μl of each concentration of YN17 (final concentrations of 0.8, 0.4 and 0.2 mg/ml) was added, 3 μl of inducer ADP (ADP concentration of 150 μmol/L) was added after 15 minutes, and the maximum platelet aggregation rate (%) was recorded within 300 s. The positive control group was added with 30 μl clopidogrel (final concentration of 0.4 mg/ml).

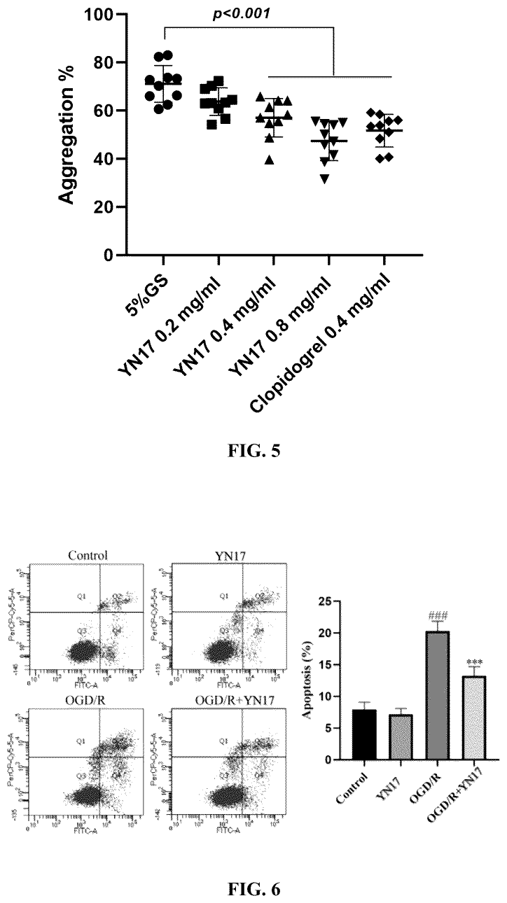

Result: ADP (30 μl, 150 μmol/L) can induce platelet aggregation in the rats, and the maximum aggregation rate within 5 minutes was 71.10%. YN17 0.2, 0.4 and 0.8 mg/ml (final concentration) can significantly inhibit the ADP-induced maximum platelet aggregation rate (p<0.001) within the range of 0.2-0.8 mg/ml and in a dose-dependent manner. Clopidogrel (0.4 mg/ml) significantly inhibited ADP-induced platelets in the rats. The aggregation rate (p<0.001) of clopidogrel showed no significant difference in the strength of action compared with YN17 0.4 mg/ml. The data are shown in Table 2 and FIG. 5.

TABLE 2
Effect of YN17 on platelet aggregation in rats (Mean ± SD)

| | | Platelet maximum |
|---|---|---|
| Group | n | aggregation rate % |
| 5% glucose control group | 10 | 71.10 ± 7.61 |
| YN17 0.2 mg/ml group | 10 | 63.76 ± 5.72 |
| YN17 0.4 mg/ml group | 10 | 57.01 ± 8.00*** |
| YN17 0.8 mg/ml group | 10 | 47.34 ± 8.10*** |
| Clopidogrel 0.4 mg/ml group | 10 | 51.68 ± 6.77*** |

Compared with the 5% glucose control group,
***p < 0.001.

### Example 3: Protective Effect of a Chalcone Glycoside Compound on Cellular Oxygen-Glucose Deprivation/Reperfusion Injury

### Test Method

Establishment of cellular oxygen-glucose deprivation/reperfusion (OGD/R) models. In vivo ischemia was simulated by incubation in a three-gas incubator (95% N₂, 5% CO₂) using sugar-free culture medium for 8-h culture, followed by transfer to a normal CO₂ incubator for 12-h culture to simulate reperfusion.

The effect of YN17 on OGD/R-induced apoptosis of H9c2 cells (YN17 20, 40 and 80 μg/ml can improve the survival rate of cells in different degrees. In the later mechanism research, a dose of 80 μg/ml was chosen for the study.)

Hoechst 33342 staining method for cell apoptosis detection. H9c2 cardiomyocytes were inoculated into a 6-well plate at a density of 2×10⁵ cells/well. When the cells were completely adhered to the wall and grew to an appropriate degree of confluence, the cells were divided into a control group, a YN17 80 μg/ml group, a model group, and an OGD/R+80 μg/ml group. In the blank group, 2 mL of a fresh culture medium was added to each well and cultured in a CO₂ incubator. The same amount of sugar-free DMEM was added to each well in the model group. After culture in a three-gas incubator for 8 h, the sugar-free DMEM was replaced with a fresh culture medium and transferred to a CO₂ incubator for additional 12-h culture. The administration group was pretreated with different concentrations of YN17 for 24 h before OGD/R modeling. Other operations were the same as the model group. After the treatment of each group, staining was performed according to the instructions of the Hoechst 33342 staining solution, and the nucleus morphology and fluorescence intensity of each group were observed under a fluorescence microscope.

Flow cytometry for cell apoptosis detection. H9c2 cardiomyocytes were inoculated into a 6-well plate at a density of 2×10⁵ cells/well. When the cells were completely adhered to the wall and grew to an appropriate degree of confluence, the cells were grouped and treated according to the method of “Hoechst 33342 staining method for cell apoptosis detection”. After the treatment of each group, staining was performed according to the instructions of the Annexin V-FITC cell apoptosis detection kit, and cell apoptosis was detected by a flow cytometer.

Result: after Hoechst 33342 staining, there were no obvious apoptosis characteristics in the control group and YN17 group, and the nuclei were large and full, showing uniform weak blue fluorescence. Compared with the control group, the OGD/R group showed obvious apoptosis characteristics, manifested as cell nucleus shrinkage and deformation, varying sizes, nuclear chromatin pyknosis and accumulation, or blocky and ruptured changes, and fluorescence enhancement and densification. However, after YN17 treatment, the number of densely and heavily stained cell nuclei can be significantly reduced, demonstrating an inhibitory effect on apoptosis.

Flow cytometry was used to detect cell apoptosis, as shown in FIG. 6. The control group had an apoptosis rate of 7.9%, while the model group had an apoptosis rate of 20.3%, which was statistically significant compared with the control group (P<0.001). However, after YN17 treatment, the apoptosis rate was reduced to 13.3%, indicating that YN17 can significantly alleviate OGD/R-induced cell apoptosis (P<0.001).

### Example 4: Evaluation of the Effect of a Chalcone Glycoside Compound on Ischemia-Reperfusion in Rats

### (1) Tests for Different Doses of YN17:

### Test Method

Establishment of cerebral ischemia-reperfusion rat models. Rats were anesthetized with pentobarbital sodium (45 mg/kg), the neck skin was prepared, the surgical skin was disinfected with iodophor, the neck skin was cut in the middle, the neck muscles were separated, and the left common carotid artery (CCA) was separated until the branches of the internal carotid artery (ICA) and external carotid artery (ECA) were exposed. The proximal end of the common carotid artery was ligated, the distal end of the common carotid artery was clamped with an artery clip, an incision was made between the two ligated ends of the common carotid artery, the MCAO suture was inserted, and the suture was loosely ligated to the common carotid artery. The MCAO suture was inserted into the internal carotid artery, so that the suture reached the branches of the internal carotid artery to the middle cerebral artery (MCA), blocking the blood supply of the MCA and causing ischemia, the MCAO suture was fixed, and the surgical incision was sutured. After 1 h of ischemia, the MCAO suture was withdrawn into the common carotid artery to restore blood supply to the middle cerebral artery.

Drug administration. After the surgery, the rats were immediately injected with YN17 (5 mg/kg, 10 mg/kg and 20 mg/kg) via the tail vein, while the positive control group was injected with 3 mg/kg of edaravone via the tail vein. The rats were euthanized under anesthesia 24 hours after drug administration.

Animal neurobehavioral assessment. 24 hours after administration, the activity of the rats after landing was observed, and the behavior disorders of the rats were scored. The scoring method was as follows:
0 points: no loss of neurological function and normal activity;
1 point: failure to fully extend the left forepaw on the opposite side of the lesion;
2 points: failure of the paralyzed side to resist the thrust from the opposite side;
3 points: circling to the left side when crawling (tail-chasing sign);
4 points: falling to the paralyzed side when shaking or walking; and
5 points: trance or loss of consciousness.

Determination of the extent of cerebral infarction. The rats were euthanized under anesthesia 24 hours after drug administration. The brain was removed and rinsed with normal saline at 4° C. The brain was frozen at −80° C. for 10 min, and coronal slices with a thickness of 2 mm were cut from posterior to anterior, incubated in a 1% TTC solution for 10 min at 37° C., and fixed with formalin overnight after staining. The photos of the brain slices were taken and saved. The area of each brain slice and the area of the infarcted area were measured by Image Pro software, and the percentage of the area of focuses of infarct to the total area was calculated.

Results: 24 h after modeling, the rats in the cerebral ischemia-reperfusion model group showed obvious behavior disorders, such as circling to the left side when crawling (tail-chasing sign), falling to the paralyzed side when shaking or walking, and other symptoms. After intravenous injection of 10 mg/kg and 20 mg/kg of YN17, the symptoms of behavior disorders were significantly improved (p<0.01). Intravenous injection of 3 mg/kg of edaravone can also significantly improve behavior disorders induced by cerebral ischemia-reperfusion in the rats (p<0.05).

The TTC staining results of brain tissue showed that obvious infarction focuses appeared in the brain tissue of the rats in the model group, and intravenous injection of 5 mg/kg, 10 mg/kg and 20 mg/kg of YN17 showed dose-dependent inhibition of infarction focuses in brain tissue. Compared with the model group, the compound 10 mg/kg and 20 mg/kg groups showed a significant reduction in infarction focuses in the brain tissue of the rats (p<0.01). The positive control, intravenous injection of 3 mg/kg of edaravone, can also significantly inhibit brain tissue injury induced by ischemia-reperfusion (p<0.05).

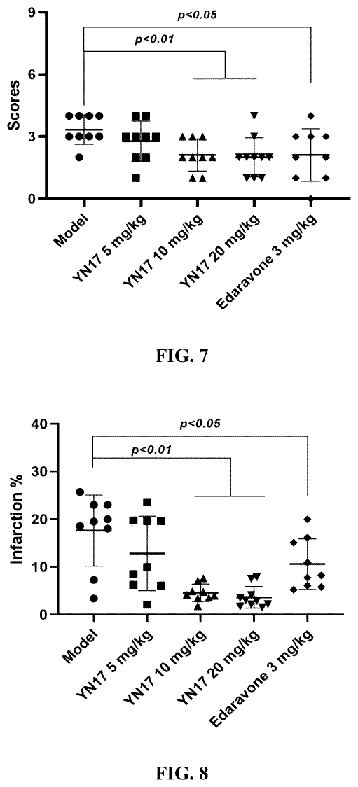

24 h after modeling, the rats in the cerebral ischemia-reperfusion model group showed significantly decreased contents of GSH-px and SOD in serum (p<0.05-0.001), and a significantly increased content of MDA (p<0.05). 5 mg/kg, 10 mg/kg and 20 mg/kg of YN17 can significantly increase the content of GSH-px in serum (p<0.001); and 10 mg/kg and 20 mg/kg of YN17 can significantly increase the content of SOD in serum (p<0.001). 3 mg/kg of edaravone can also significantly increase the contents of GSH-px and SOD in serum (p<0.001). Please see Table 3 and FIGS. 7 to 8.

TABLE 3
Effect of YN17 on cerebral ischemia-reperfusion (Mean ± SD)

| | | | Percentage of whole |
|---|---|---|---|
| | Number of | Symptom | brain tissue infarction |
| Group | animals | score | (%) |
| Blank control group | 10 | 0 | 0 |
| Model group | 9 | 3.3 ± 0.7 | 17.61 ± 7.45 |
| YN17 5 mg/kg group | 9 | 2.8 ± 1.0 | 12.80 ± 7.80 |
| YN17 10 mg/kg group | 9 | 2.1 ± 0.8** | 4.58 ± 1.82** |
| YN17 20 mg/kg group | 10 | 2.0 ± 0.9** | 3.62 ± 2.28** |
| Edaravone 3 mg/kg | 9 | 2.1 ± 1.3* | 10.58 ± 5.31* |
| group | | | |

Compared with the model group,
*p < 0.05,
**p < 0.01.

### (2) Comparative Tests on Calcium Salt YN17 and Sodium Salt YN17-ST:

The models were established and administered according to the similar method described above (YN17 (10 mg/kg) and YN17-ST (10 mg/kg) were injected into the tail vein immediately after blood supply to the middle cerebral artery of the rats was restored, and 5% glucose was injected into the tail vein in the model group. The rats were euthanized under anesthesia 24 hours after drug administration, and the extent of cerebral infarction was measured. The results were as follows:

TABLE 4
Protective effects of YN17 and YN17-ST on cerebral
ischemia-reperfusion in rats (Mean ± SEM)

| | | Percentage of hemispheric |
|---|---|---|
| Group | N | tissue infarction (%) |
| Model | 8 | 51.37 ± 2.66 |
| YN17-ST 10 mg/kg | 8 | 23.35 ± 7.37** |
| YN17 10 mg/kg | 11 | 20.47 ± 3.56*** |

Compared with the model group:
**p < 0.01.

The results showed that after 1 hour of cerebral ischemia and reperfusion in rats, the volume of cerebral infarction in hemispheric tissue was 51.37%. The cerebral infarction volumes after intravenous injection of 10 mg/kg of YN17-ST and YN17 in rats were 23.35% and 20.47%, respectively, which were both significantly lower than that of the model group.

### Attachment

### Stability Solutions (Display of Some Test Items)

| | | | Investigation |
|---|---|---|---|
| Test type | Placement condition | Investigation time | item |
| Accelerated | 40° C. ± 2° C./ | 0 months, 1 month, | Character, pH |
| test | 75% ± 5% RH | 2 months, 3 months, | value, water |
| | Placed in a 100 mL | 6 months | content, |
| | medicinal aluminum | | |
| | bottle with a rolled cap | | |
| Long-term | 25° C. ± 2° C./ | 0 months, 3 months, | calcium |
| test | 60% ± 5% RH | 6 months, 9 months, | element, |
| | Placed in a 100 mL | 12 months, 18 months, | content |
| | medicinal aluminum | 24 months | determination |
| | bottle with a rolled cap | | |

### Quality Standards (Display of Some Test Items)

[Character] Ocular estimate method: this sample was a yellow powder.

[Examination] PH value: this sample was taken and added with water to make a solution containing 5 mg per 1 ml. According to the pH value determination method (Chinese Pharmacopoeia 2020 Edition Fourth General Rule 0631), the pH value should be 5.0-7.0.

Relevant substances. Determination of relevant substances was performed according to high-performance liquid chromatography (Chinese Pharmacopoeia 2020 Edition Fourth General Rule 0512).

Test sample solution. An appropriate amount of this sample was taken, accurately weighed, dissolved by addition of a diluent, and diluted quantitatively to prepare a solution containing 1.0 mg per 1 ml. The solution was shaken well, filtered through a microporous filter membrane (0.45 μm), and the subsequent filtrate was taken.

System suitability solution. An appropriate amount of a comparative sample, and added with a diluent to prepare a solution containing 0.01 mg per 1 ml.

Chromatographic conditions. Octadecylsilane bonded silica gel was used as a filler: 0.01 mol/mL potassium dihydrogen phosphate solution (1.36 g of potassium dihydrogen phosphate was taken into a 1000 mL measuring flask, dissolved by addition of water, and diluted to the scale) was used as a mobile phase A, methanol was used as a mobile phase B, and gradient elution was performed according to the following table; the detection wavelength was 375 nm; and the injection volume was 5 μl.

| Time (minutes) | A (%) | B (%) |
|---|---|---|
| 0-50 | 95→70 | 5→30 |
| 50-60 | 70→95 | 30→5 |

System suitability requirements. In the chromatogram of the system suitability solution, the number of theoretical plates calculated based on the main peak of the comparative sample solution should not be less than 3000.

Diluent Mobile phase A-mobile phase B (95:5).

Determination method. The test sample solution and system suitability solution were accurately measured, and injected into the liquid chromatograph separately, and the chromatogram was recorded.

Limit If there were impurity peaks in the chromatogram of the test sample solution, the area of a single impurity peak shall not be greater than 0.005 times (0.5%) the total peak area, and the sum of the areas of each impurity peak shall not be greater than 0.015 times (1.5%) the total peak area. Chromatographic peaks less than 0.02% of the peak area of the main component can be ignored.

Water content This sample was taken, and according to the method for water content determination (Chinese Pharmacopoeia 2020 Edition Fourth General Rule 0832 First Method), the water content shall not exceed 15.0%.

Calcium element Determination of calcium element was performed according to inductively coupled plasma mass spectrometry (Chinese Pharmacopoeia 2020 Edition Fourth General Rule 0412).

[Content determination] Content determination was performed according to high-performance liquid chromatography (Chinese Pharmacopoeia 2020 Edition Fourth General Rule 0512).

Test sample solution. An appropriate amount of this sample was accurately weighed, and added with water to prepare a solution containing about 0.40 mg per 1 ml as the test sample solution.

Comparative sample solution. An appropriate amount of a comparative sample was accurately weighed, and added with water to prepare a solution containing 0.40 mg per 1 ml.

System suitability solution. Same as the comparative sample solution.

Chromatographic conditions. Octadecylsilane bonded silica gel was used as a filler, methanol-acetonitrile-water-phosphoric acid (26:2:72:0.04) was used as a mobile phase, and detection wavelength was 403 nm; and the injection volume was 10 μl.

System suitability requirements. The number of theoretical plates calculated based on the main peak of the comparative sample solution should not be less than 3000.

Determination method. The test sample solution and comparative sample solution were accurately measured, and injected into the liquid chromatograph separately, and the chromatogram was recorded. The content of C₂₇H₃₂O₁₆ in the test sample was calculated by the peak area according to the external standard method.

What is claimed is:

1. A chalcone glycoside compound, having a structure of: 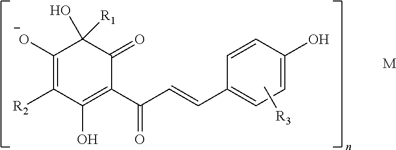
or a hydrate thereof,
wherein R₁ and R₂ are β-D-glucosyl or α-D-glucosyl; R₃ is hydrogen or hydroxyl; and M is a metal ion, and n is 1-2.

2. The chalcone glycoside compound according to claim 1, wherein M is a sodium ion or a calcium ion.

3. The chalcone glycoside compound according to claim 1, having a structure of: 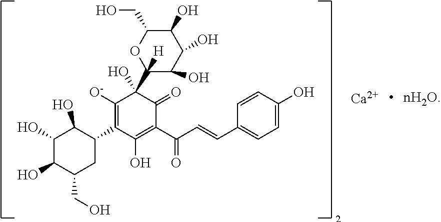
or a hydrate thereof.

4. The chalcone glycoside compound according to claim 3, wherein the compound further has an octahedral ligand stereostructure.

5. The chalcone glycoside compound according to claim 3, wherein the compound is: (S)-3,6-dihydroxy-4-((E)-3-(4-hydroxyphenyl) acryloyl)-5-oxo-6-((2R,3R,4S,5S,6R)-3,4,5-trihydroxy-6-(hydroxymethyl)tetrahydro-2H-pyran-2-yl)-2-((2S,3R,4R,5S,6R)-3,4,5-trihydroxy-6-(hydroxymethyl)tetrahydro-2H-pyran-2-yl)cyclohexyl-1,3-diene-1-hydroxycalcium or a hydrate thereof.

6. A crystal form A of a chalcone glycoside compound, having the structure according to claim 3, wherein an XRPD spectrum has diffraction peaks at 2θ=7.234 degrees, 7.866 degrees, 8.243 degrees, 8.792 degrees, 14.251 degrees, 15.791 degrees, and 16.283 degrees, and the 20 deviation is ±0.2 degrees.

7. The crystal form A of a chalcone glycoside compound according to claim 6, wherein the XRPD spectrum further has diffraction peaks at 2θ=9.959 degrees, 10.716 degrees, 12.722 degrees, 16.786 degrees, 20.764 degrees, and 23.955 degrees.

8-13. (canceled)

14. An application of a chalcone glycoside compound in preparation of a medicament for treating a vascular disease, wherein the chalcone glycoside compound is the chalcone glycoside compound according to claim 1, wherein the vascular disease is a vascular disease caused by free radical oxidation, an inflammatory mediator and platelet aggregation, comprising one or more of stroke, a coronary syndrome, a pulmonary heart disease, a peripheral vascular disease, and peripheral neuropathy.
